(19) United States
(12) Patent Application Publication
(10) Pub. No.: US 2006/0235053 A1
(43) Pub. Date: Oct. 19, 2006

(54) Agents for the treatment of lower abdominal disorders

(75) Inventors: Ursula Gebauer, Konstanz (DE); Iris Velten, Konstanz (DE); Wolfgang-Alexander Simon, Konstanz (DE)

Correspondence Address: NATH & ASSOCIATES PLLC, 112 South West Street, Alexandria, VA, 22314, US

(73) Assignee: Atlanta Pharma AG, Konstanzde (DE)

(21) Appl. No.: 10/555,055

(22) PCT Filed: May 4, 2004
(86) PCT No.: PCT/EP04/50694

§ 371 Date: Nov. 25, 2005

(30) Foreign Application Priority Data
May 6, 2003 (EP) 0300168.7
Jul. 19, 2003 (EP) 03016362.0

(51) Int. Cl. A61K 31/4439

(52) U.S. Cl. 514/338

(57) ABSTRACT

The invention relates to the use of pantoprazole in the treatment of lower abdominal disorders.

### TECHNICAL FIELD

The invention relates to the use of compounds from the class consisting of the add secretion inhibitors for the treatment of lower abdominal disorders.

### PRIOR ART

A whole series of compounds are known from the prior art which inhibit gastric acid secretion by blocking the proton pump and which have therefore also been designated as proton pump inhibitors (PPI). These compounds are suitable for the treatment of gastric and upper abdominal intestinal disorders and accordingly some of them have been approved by health authorities. In international Patent Application WO-A-03053221, an invention is described which provides methods of treating and preventing asthma, laryngitis, symptomatic gastroesophageal reflux disease, pregnancy-induced gastroesophageal reflux disease, noncardiac chest pains, coughing, apnea, dyspepsia, inflammatory bowel disease, irritable bowel syndrome, gastritis, stress ulcers, bleeding peptic ulcers, acute gastrointestinal bleeding, infectious enteritis, collagenous colitis, lymphocytic colitis, chronic diarrhea in immunocompromised patients, esophageal ulcers in immunocompromised patients, idiopathic gastric acid hypersecretion, gastroparesis, gastrointestinal motility disorders, Zollinger-Ellison syndrome, short bowel syndrome, emesis, regurgitation, early satiety, chronic sore throat, abdominal pain, abdominal bloating, nausea, sour stomach, diarrhea, constipation, bacterial infections, refractory ulcers, gastrointestinal disorders induced by NSAIDs, Barrett's esophagus, gastrointestinal disorders caused by steroids, gastrointestinal disorders induced by cholinergic compounds, and fungal or viral-induced ulcers in the gastrointestinal tract, by administering a therapeutically effective amount of at least one proton pump inhibitor to a patient in need thereof. The invention described in intentional Patent Application WO-A-03053221 also provides on demand relief of symptoms associated with gastroesophageal reflux disease (GERD), and provides relief from symptoms caused by the consumption of excessive amounts of food and/or alcohol by administering a therapeutically effective amount of at least one proton pump inhibitor to a patent in need thereof. The invention described in intentional Patent Application WO-A-03053221 also provides methods for treating parasitic infections, such as malaria, by administering a therapeutically effective amount of at least one proton pump inhibitor to a patient in need thereof. —Chinese Patent Application 1369491 relates to the salts of the levo (−) and dextro (+) enantiomers of the peptic ulcer resistant medicine (±)5-difluoromethoxy-[[3,4-dimethoxy-2-pyridyl)methyl]sulfinyl]-1H-benzimidazole, i.e. to the potassium (or sodium, or magnesium, or calcium, or zinc) salt of S-(−)-Pantoprazole (or R-(+)-Pantoprazole). —In U.S. Pat. No. 6,156,771, a method of alleviating a lower GI symptom in a human patient afflicted with a lower GI disorder and a method of treating a human patient afflicted with a lower GI disorder, including, for example, a patent afflicted with irritable bowel syndrome or a patient afflicted with functional diarrhea, are provided. Each of these methods comprises inhibiting gastric secretion by the patient, such as by administering to the patient a pharmaceutical preparation comprising an effective amount of an inhibitor of gastric secretion, such as a proton pump inhibitor.

### DESCRIPTION OF THE INVENTION

Surprisingly, it has now been found that the proton pump inhibitors, whose original field of use is the treatment of gastric and upper abdominal intestinal disorders, are particularly suitable for the treatment of lower abdominal disorders.

The invention thus relates in a first aspect to the use of proton pump inhibitors in the treatment of lower abdominal disorders.

Proton pump inhibitors are designated as those substances, which inhibit gastric acid secretion by blocking the proton pump, i.e. substances which bind covalently to the H⁺/K⁺-ATPase, the enzyme responsible for gastric add secretion. These include in particular active compounds having a 2-[(2-pyridinyl)methylsulphinyl-1H-benzimidazole skeleton or related skeletons, where these skeletons may be substituted in various different ways. The term “proton pump inhibitor” according to the invention comprises not only the active compounds as such, but also their pharmacologically acceptable salts, solvates (in particular hydrates), etc.

Examples of proton pump inhibitors which may be mentioned are those described and claimed in the patent applications and patents below: DE-A-3531487, EP-A-0 005 129, EP-A-0 124 495, EP-A-0 166 287, EP-A 0 174 726, EP-A-0 184 322, EP-A-0 254 588, EP-A-0 261 478, EP-A-0 268 956, EP-A-0 434 999 and WO-A-9523149. Examples which may be mentioned here are the compounds 2-[2-(N-isobutyl-N-methylamino)benzylsulphinyl]benzimidazole (INN: leminoprazole), 2-(4-methoxy-6,7,8,9-tetrahydro-5H-cyclohepta[b]pyridin-9-ylsulphinyl)-1H-benzimidazole (INN: nepaprazole), 2-(4-methoxy-3-methylpyridin-2-ylmethylsulphinyl)5-pyrrol-1-yl-1H-benzimidazole (IY-81149), 5-methoxy-2-[(4-meth-oxy-3,5-dimethyl-2-pyridinyl)methylsulphinyl]-1H-imidazo[4,5-b]pyridine (tenatoprazole), especially 5-methoxy-2-[(4-methoxy-3,5-dimethyl-2-pyridinyl)methylsulphinyl]-1H-benzimidazole (INN: omeprazole), 5-methoxy-2-[(S)-[(4-methoxy-3,5-dimethyl-2-pyridinyl)methyl]sulphinyl]-1H-benzimidazole (INN: esomeprazole), 2-[(3-methyl-4-(2,2,2-trifluoroethoxy)-2-pyridinyl)methylsulphinyl]-1H-benzimi-dazole (INN: lansoprazole) and 2-{[4-(3-methoxypropoxy)-3-methylpyridin-2-yl]-methylsulphinyl}-1H-benzimidazole (INN: rabeprazole) and in particular 5-difluoromethoxy-2-[(3,4-dimethoxy-2-pyridinyl)methylsulphinyl]-1H-benzimidazole (INN: pantoprazole) and (−)-5-difluoromethoxy-2-[(3,4-dimethoxy-2-pyridinyl)methylsulphinyl]-1H-benzimidazole [INN: (−)-pantoprazole].

The proton pump inhibitors are present as such or in the form of their salts with bases. Examples of salts with bases which may be mentioned are sodium, potassium, magnesium or calcium salts. If the proton pump inhibitors or their salts are isolated in crystalline form, the crystals may contain variable amounts of solvent. Correspondingly, according to the invention, the term “proton pump inhibitor” also includes all solvates, in particular all hydrates, of the proton pump inhibitors and their salts. Particularly preferred salts or hydrates of proton pump inhibitors, which may be mentioned are pantoprazole-sodium sesquihydrate (=pantoprazole-sodium×1.5 H₂O), (−)-pantoprazole-sodium sesquihydrate, pantoprazole-magnesium dihydrate, omeprazole-magnesium, omeprazole-magnesium tetrahydrate, esomeprazole-magnesium and esomeprazole-magnesium tetrahydrate.

Lower abdominal disorders to be treated, which may be mentioned in particular are the irritable bowel syndrome (IBS), lower abdominal pain/discomfort (particularly symptomatology), inflammatory bowel diseases such as Colitis ulcerosa (ulcerative colitis) and Morbus Crohn (Crohn's disease, regional enteritis, enterocolitis, ileitis, terminal ileitis) and menstrual symptoms.

The invention relates in a further aspect to the use of proton pump inhibitors for the treatment of patients who are suffering from a lower abdominal disorder.

The invention further relates to a method for the treatment of lower abdominal disorders, which consists in administering to a patient who needs such a treatment an effective amount of a proton pump inhibitor.

The invention further relates to the use of proton pump inhibitors for the production of medicaments for the treatment of lower abdominal disorders.

The invention further relates to a pharmaceutical preparation for the treatment of lower abdominal disorders, which contains a proton pump inhibitor as active compound.

The invention further relates to a ready-to-use medicament, comprising a proton pump inhibitor as active compound, which contains a reference to the fact that this ready-to-use medicament can be employed for the treatment of lower abdominal disorders.

in particular, it has been found that the proton pump inhibitor pantoprazole, whose original field of use is the treatment of gastric and upper abdominal intestinal disorders, is—due to its long duration of action—particularly suitable for the treatment of lower abdominal disorders.

The invention thus relates in a preferred aspect to the use of pantoprazole in the treatment of lower abdominal disorders.

According to the invention, “pantoprazole” comprises not only the active compound as such, but also its enantiomers, i.e. (R)- and (S)-pantoprazole, as well as pharmacologically acceptable salts, solvates (in particular hydrates), etc. of pantoprazole, (R)-pantoprazole and (S)-pantoprazole.

Examples of pharmacologically acceptable salts, which may be mentioned, are sodium, potassium, magnesium or calcium salts. If pantoprazole or its salts is isolated in crystalline form, the crystals may contain variable amounts of solvent.

Particularly preferred salts or hydrates of pantoprazole which may be mentioned are pantoprazole-sodium sesquihydrate (=pantoprazole-sodium×1.5 H₂O), (S)-pantoprazole-sodium sesquihydrate, pantoprazole-magnesium dihydrate and (S)-pantoprazole-magnesium dihydrate.

The invention relates in a preferred aspect to the use of pantoprazole for the treatment of patients who are suffering from a lower abdominal disorder.

The invention further particularity relates to a method for the treatment of lower abdominal disorders, which consists in administering to a patient who needs such a treatment an effective amount of panto-prazole.

The invention further particularly relates to the use of pantoprazole for the production of medicaments for the treatment of lower abdominal disorders.

The invention further particularly relates to a pharmaceutical preparation for the treatment of lower abdominal disorders, which contains pantoprazole as active compound.

The invention further particularly relates to a ready-to-use medicament, comprising pantoprazole as active compound, which contains a reference to the fact that this ready-to-use medicament can be employed for the treatment of lower abdominal disorders.

Commercial Utility

According to the invention, the proton pump inhibitors, in particular pantoprazole, are employed for the treatment of lower abdominal disorders in the form of ready-to-use medicaments. These medicaments are prepared by methods known per se and familiar to the person skilled in the art. As medicaments, the proton pump inhibitors are either used here as such, or preferably in combination with suitable pharmaceutical excipients or vehicles in the form of tablets, coated tablets, capsules, suppositories, patches (e.g. as TTS), emulsions, suspensions or solutions, the active compound content advantageously being between 0.1 and 95% and it being possible by means of the appropriate choice of the excipients and vehicles to achieve a pharmaceutical administration form adapted exactly to the active compound and/or to the desired onset of action and/or to the duration of action (e.g. a sustained release form or an enteric form).

The person skilled in the art is familiar on the basis of his/her expert knowledge with which excipients or vehicles are suitable for the desired pharmaceutical formulations. Besides solvents, gel-forming agents, suppository bases, tablet excipients and other active compound carriers, it is possible to use, for example, antoxidants, dispersants, emulsifiers, antifoams, taste corrigents, preservatives, solubilizers, colorants or, in particular, permeation promoters and complexing agents (e.g. cyclodextrins).

The active compounds can be administered orally, parenterally or percutaneously.

in general, it has proved advantageous in human medicine to administer the proton pump inhibitor in a daily dose of, in particular, 0.1 to 1.5 mg/kg of body weight, if appropriate in the form of a number of, preferably 1 to 2, individual doses to achieve the desired result in the case of a parenteral treatment, similar or (in particular in the case of the intravenous administration of the active compounds) as a rule lower dosages can be used. The determination of the optimal dosage and manner of administration of the active compounds necessary in each case can be carried out easily by any person skilled in the art on the basis of his/her expert knowledge.

The invention further relates to a pharmaceutical preparation for the treatment of lower abdominal disorders, which in an individual dose (tablet, capsule, etc.) contains a proton pump inhibitor, in particular pantoprazole, as active compound in a dose of between 5 and 100, advantageously between 10 and 60, in particular between 20 and 40 mg.

If the proton pump inhibitors, in particular pantoprazole, are to be employed for the treatment of lower abdominal disorders, the pharmaceutical preparations can also contain one or more pharmacologically active constituents of other pharmaceutical groups. Examples, which may be mentioned are: tranquilizers (for example from the group consisting of the benzodiazepines, e.g. diazepam), spasmolytics (e.g. bietamiverine or camylofine), anticholinergics (e.g. oxyphencyclimine or phencarbamide), local anesthetics (e.g. tetracaine or procaine), and optionally also enzymes, vitamins or amino acids.

The combination of proton pump inhibitors, in particular pantoprazole, with those pharmaceuticals, which are customarily employed for the treatment of lower abdominal disorders is to be particularly emphasized in this context Examples, which may be mentioned, are pharmaceuticals for the treatment of Morbus crohn and Colitis ulcerosa, such as aminosalicylates (e. g. mesalazine, olsalazine and sulfosalazine), glucocorticoids (e.g. betamethason, budesonide, hydrocortisone acetate, methylprednisolone, prednisolone and prednisone) and immunosuppressive agents (e. g. azathioprin, cyclosporin, methotrexat and infliximab), pharmaceuticals for the treatment of diarrhoeas (e. g. colestyramine and loperamide) and pharmaceuticals for the treatment of IBS (e. g. tegaserod).

“Combination” within the meaning of the present invention is to be understood as meaning that the individual components can be administered simultaneously (in the form of a combination medicament—fixed combination) or more or less simultaneously, respectively in succession (from separate pack units—free combination; directly in succession or else alternatively at a relatively large time interval) in a manner which is known per se and customary. As an example, one therapeutic agent could be taken in the morning and one later in the day. Or in another scenario, one therapeutic agent could be taken once daily and the other once weekly or only once monthly.

Simultaneous administration preferably is accomplished by administering to the subject in need thereof, for example, a single intravenous injection/infusion having a fixed ratio of each therapeutic agent. More or less simultaneous administration or administration in succession of each therapeutic agent can be effected by any appropriate route, inducing, but not limited to, oral mutes, intravenous routes, intramuscular routes, and by Infusion techniques. The therapeutic agents can be administered by the same route or by different routes. For example, a first therapeutic agent of the combination selected may be administered by intravenous or subcutaneous injection while the other therapeutic agent of the combination may be administered orally. The sequence in which the therapeutic agents are administered is not narrowly critical.

The therapeutic agent(s) according to the invention may be administered in a variety of forms. These include, for example, liquid, semi-solid and solid dosage forms, such as liquid solutions (e.g., injectable and infusible solutions), dispersions or suspensions, tablets, pills, powders, liposomes or suppositories. The preferred form depends on the intended mode of administration and therapeutic application.

1. (canceled)

2. (canceled)

3. A method of treating a lower abdominal disorder in a patient comprising administering to a patient in need thereof a therapeutically effective amount of a proton pump inhibitor, or a hydrate, solvate, salt, hydrate of a salt or solvate of a salt thereof.

4. (canceled)

5. (canceled)

6. A ready-to-use medicament comprising a proton pump inhibitor as active compound, or a hydrate, solvate, salt, hydrate of a salt or solvate of a salt thereof, and a reference to the fact that it can be employed for the treatment of lower abdominal disorders.

7. A method according to claim 3, wherein said proton pump inhibitor is a compound selected from the group consisting of 5-methoxy-2-[(4-methoxy-3,5-dimethyl-2-pyridinyl)methyl-sulphinyl)-1H-benzimidazole (INN: omeprazole), 5-methoxy-2-[(S)-[(4-methoxy-3,5-dimethyl-2-pyridinyl)methyl]sulphinyl]-1H-benzimidazole (INN: esomeprazole), 2-[3-methyl-4-(2,2,2-trifluoroethoxy)-2-pyridinyl)methylsulphinyl]-1H-benzimidazole (INN: lansoprazole), 2-{[4-(3-methoxypropoxy)-3-methylpyridin-2-yl]-methylsulphinyl}-1H-benzimidazole (INN: rabeprazole), 5-difluoromethoxy-2-[(3,4-dimethoxy-2-pyridinyl)methylsulphinyl]-1H-benzimidazole (INN: pantoprazole), (−)-5-difluoromethoxy-2-[(3,4-dimethoxy-2-pyridinyl)methylsulphinyl]-1H-benzimidazole [INN: (−)-pantoprazole] and the hydrates, solvates, salts, hydrates of the salts and solvates of the salts thereof.

8. The method according to claim 7, wherein said proton pump inhibitor is pantoprazole or a pharmacologically acceptable hydrate, solvate, salt, hydrate of a salt or solvate of a salt thereof.

9. The method according to claim 7, wherein said proton pump inhibitor is racemic pantoprazole or a pharmacologically acceptable hydrate, solvate, salt, hydrate of a salt or solvate of a salt thereof.

10. The method according to claim 7, wherein said proton pump inhibitor is (S)-pantoprazole or a pharmacologically acceptable hydrate, solvate, salt, hydrate of a salt or solvate of a salt thereof.

11. The method according to claim 7, wherein said proton pump inhibitor is pantoprazole-sodium or a hydrate thereof.

12. The method according to claim 7, wherein said proton pump inhibitor is pantoprazole-magnesium or a hydrate thereof.

13. The method according to claim 7, wherein said proton pump inhibitor is (S)-pantoprazole-magnesium or a hydrate thereof.

14. The method according to claim 3, wherein said lower abdominal disorder is selected from the group consisting of irritable bowel syndrome (IBS), lower abdominal pain/discomfort, symptomatology inflammatory bowel disease, (ulcerative colitis,) Crohn's disease, regional enteritis, enterocolitis, ileitis, terminal ileitis, and menstrual symptoms.

15. (canceled)

16. A method of treating a lower abdominal disorder in a patient comprising administering to a patient in need thereof a therapeutically effective amount of pantoprazole, or a hydrate, solvate, salt, hydrate of a salt or solvate of a salt thereof.

17. (canceled)

18. (canceled)

19. A ready-to-use medicament comprising pantoprazole as active compound, or a hydrate, solvate, salt, hydrate of a salt or solvate of a salt thereof, and a reference to the fact that it can be employed for the treatment of lower abdominal disorders.

20. A ready-to-use medicament for the treatment of lower abdominal disorders comprising pantoprazole, or a hydrate, solvate, salt, hydrate of a salt or solvate of a salt thereof, as one active compound and a second active compound selected from aminosalicylates glucocorticoids, immunosuppressive agents pharmaceuticals for the treatment of diarrhoeas and pharmaceuticals for the treatment of IBS.

21. A ready-to-use medicament according to claim 19, wherein the lower abdominal disorder is selected from the group consisting of irritable bowel syndrome (IBS), lower abdominal pain/discomfort, symptomatology inflammatory bowel disease, ulcerative colitis, Crohn's disease, regional enteritis, enterocolitis, ileitis, terminal ileitis, and menstrual symptoms.

22. (canceled)

23. A ready-to-use medicament according to claim 6, wherein said proton pump inhibitor is a compound selected from the group consisting of 5-methoxy-2-[(4-methoxy-3,5-dimethyl-2-pyridinyl)methylsulphinyl]-1H-benzimidazole (INN: omeprazole), 5-methoxy-2-[(S)-[(4-methoxy-3,5-dimethyl-2-pyridinyl)methyl]sulphinyl]-1H-benzimidazole (INN: esomeprazole), 2-[3-methyl-4-(2,2,2-trifluoroethoxy)-2-pyridinyl)methylsulphinyl]-1H-benzimidazole (INN: lansoprazole), 2-{[4-(3-methoxypropoxy)-3-methylpyridin-2-yl]-methylsulphinyl}-1H-benzimidazole (INN: rabeprazole), 5-difluoromethoxy-2-[(3,4-dimethoxy-2-pyridinyl)methylsulphinyl]-1H-benzimidazole (INN: pantoprazole), (−)-5-difluoromethoxy-2-[(3,4-dimethoxy-2-pyridinyl)methylsulphinyl]-1H-benzimidazole [INN: (−)-pantoprazole] and the hydrates, solvates, salts, hydrates of the salts and solvates of the salts thereof.

24. The ready-to-use medicament according to claim 19, wherein said proton pump inhibitor is racemic pantoprazole or a pharmacologically acceptable hydrate, solvate, salt, hydrate of a salt or solvate of a salt thereof.

25. The ready-to-use medicament according to claim 19, wherein said proton pump inhibitor is (S)-pantoprazole or a pharmacologically acceptable hydrate, solvate, salt, hydrate of a salt or solvate of a salt thereof.

26. The ready-to-use medicament according to claim 19, wherein said proton pump inhibitor is pantoprazole-sodium or a hydrate thereof.

27. The ready-to-use medicament according to claim 19, wherein said proton pump inhibitor is pantoprazole-magnesium or a hydrate thereof.

28. The ready-to-use medicament according to claim 19, wherein said proton pump inhibitor is (S)-pantoprazole-magnesium or a hydrate thereof.

29. The ready-to-use medicament according to claim 6, wherein said lower abdominal disorder is selected from the group consisting of irritable bowel syndrome (IBS), lower abdominal pain/discomfort, symptomatology inflammatory bowel disease, ulcerative colitis, Crohn's disease, regional enteritis, enterocolitis, ileitis, terminal ileitis, and menstrual symptoms.

30. The ready-to-use medicament according to claim 20, wherein the second active compound is selected from the group consisting of mesalazine, olsalazine, sulfosalazine, betamethason, budesonide, hydrocortisone acetate, methylprednisolone, prednisolone, prednisone, azathioprin, cyclosporin, methotrexat, infliximab, colestyramine, loperamide and tegaserod.
